(19) United States
(12) Patent Application Publication
(10) Pub. No.: US 2010/0209489 A1
(43) Pub. Date: Aug. 19, 2010

(54) Formulations of desvenlafaxine

(75) Inventors: Likan Liang, Boyds, MD (US); Padmanabh P. Bhatt, Rockville, MD (US); Hua Wang, Clarksville, MD (US)

Correspondence Address: FOLEY AND LARDNER LLP;SUITE 500, 3000 K STREET NW, WASHINGTON, DC, 20007, US

(73) Assignee: Supernus Pharmaceuticals, Inc.

(21) Appl. No.: 12/656,539

(22) Filed: Feb. 2, 2010

Related U.S. Application Data
(60) Provisional application No. 61/149,852, filed on Feb. 4, 2009.

(51) Int. Cl. A61K 31/135; A61K 9/127; A61K 9/14; A61K 9/22; A61K 9/00; A61P 25/24

(52) U.S. Cl. 424/450; 514/653; 424/489; 424/468; 424/484; 424/400

(57) ABSTRACT

Controlled release formulations of active compounds with pH-dependent solubility are provided. The formulations comprise solubility modulators which minimize the influence of environment on the solubility of the active compounds.

### CROSS-REFERENCE TO RELATED APPLICATIONS

This application claims priority from U.S. Provisional Application No. 61/149,852, filed Feb. 4, 2009.

### BACKGROUND OF THE INVENTION

Racemic desvenlafaxine is an active metabolite of venlafaxine. One of its enantiomers, (−)-O-desmethyl venlafaxine HCl monohydrate, is thought to be more tolerable than the racemic mixture. As a selective serotonin and norepinephrine reuptake inhibitor, desvenlafaxine is currently approved for the treatment of major depressive disorder (MDD). The recommended dose is 50 mg once daily with or without food. A dose of 50-400 mg/day in clinical studies is shown to be effective, but no additional benefit was observed at doses higher than 50 mg/day, and adverse events and discontinuations were more frequent at higher doses.

The solubility of (−)-O-desmethyl venlafaxine HCl monohydrate is highly dependent on pH. At low pH, for example pH 1.0, the solubility is in the range of a few hundred mg/mL; at about pH 6.0, the solubility is less than 100 mg/mL; at higher pH, e.g. close to the physiological pH, the solubility dramatically decreases to less than 1 mg/mL. The significant pH dependency of solubility presents challenges for the development of controlled release formulations of desvenlafaxine or derivatives or parent compounds thereof, and for obtaining consistent dissolution profiles.

### SUMMARY OF THE INVENTION

The current invention provides controlled release formulations of active compounds with pH-dependent solubility, the formulations comprising solubility modulators which minimize the influence of environmental pH change on the solubility of the active compounds.

In one embodiment, controlled release formulations are matrix formulations. In other embodiments, the controlled release formulations are osmotic formulations.

In a further embodiment, the pharmaceutically active compound with a pH-dependent solubility is desvenlafaxine. In a yet further embodiment, the desvenlafaxine is (−)-O-desmethyl venlafaxine HCl monohydrate.

The invention further provides methods to delay the release (lag time) of the active compounds to avoid certain side effects related to the gastric exposure of the active compounds.

### BRIEF DESCRIPTION OF THE DRAWINGS

FIG. 1 shows dissolution profiles of various osmotic formulations of (−)-O-desmethyl venlafaxine HCl monohydrate.

FIG. 2 shows dissolution profiles of various osmotic formulations of (−)-O-desmethyl venlafaxine HCl monohydrate.

### DEFINITIONS

For the purposes of this application, desvenlafaxine includes desvenlafaxine and related compounds, for example venlafaxine, derivatives of desvenlafaxine and venlafaxine, their racemic mixtures or single isomers, as free bases and/or salts, hydrates, and morphological forms.

Solubility regulating agents are defined as agents that minimize the solubility differences of the active compounds over a range of pH.

Environment regulating agents are those agents which minimize the changes in the environment or minimize the impact of the environmental changes, such as pH changes.

For the purposes of this application, solubility regulating agents and/or environment regulating agents may be referred to as solubility modulators.

Release regulating agents are defined as agents that regulate the release profile of the drug from the dosage form.

A formulation with a “consistent dissolution profile” is defined as a formulation with a similarity factor f2>50.

Unless otherwise specified, “a” or “an” means “one or more.”

### DETAILED DESCRIPTION OF THE PREFERRED EMBODIMENTS

The current invention provides controlled release pharmaceutical formulations with a consistent dissolution profile comprising: (1) at least one pharmaceutical agent with a pH-dependent solubility; and (2) at least one solubility modulator. Typically, the concentration of active compound(s) in such formulations constitutes from 1% wt to 70% wt, and the amount of at least one solubility modulator constitutes from 1% wt to 50% wt.

The solubility modulators for the active compounds with pH-dependent solubility. are used to adjust and control the effective solubility of the active compounds over a range of pH. The solubility modulators are not thought to significantly modify the intrinsic solubility of the active compounds. Rather, it is believed that the solubility modulators allow the solubility to stay within a preferred range.

The solubility modulators may exert their action either by interacting with the active agent or by creating a microenvironment around the active agent. By stabilizing the solubility, the modulators can help to improve the reproducibility of dissolution profiles.

A variety of compounds may be used as solubility modulators. Without placing any limitations thereon, these compounds may be selected from complexing agents, buffering agents, acids and/or salts thereof, such as citric acid or salt, tartaric acid or salt, malic acid or salt; acid-base complexes, acidic polymers, acrylic acid polymers and copolymers, cationic polymers, ion exchange resins, lipids including phospholipids and their analogues and derivatives, ionic surfactants, peptides (including oligo-, polypeptides), carbohydrates including cationic or non-ionic carbohydrates, polycarbohydrates and oligocarbohydrates such as cyclodextrin or derivatives thereof; non-ionic surfactants, non-ionic polymers with donor/receptor functional groups (such as polyethylene glycols, polyvinyl alcohols, polyvinyl pyrrolidones, polyethyleneimines), poly(Y-benzyl L-glutamate), lactide and glycolide polymers and copolymers and their derivatives, polyethyleneglycol-poly(ortho ester) block copolymers, poly(styrene)-poly(methylmethacrylate) block copolymers, PEO-PPO-PEO block copolymers, poly(2-phenoxyethyl vinyl ether)-poly(2-methoxyethyl vinyl ether)) block copolymers, carbohydrate-substituted porphyrins, block polymers comprising polyacetylene, block polymers comprising poly(p-phenylene vinylene), block polymers comprising poly(p-phenylene), block polymer comprising polypyrrole, block polymers comprising polythiophene, alginic acid complexes, among others.

Self-assembling agents forming micro-structures/nano-structures (e.g. micelles, reverse micelles, microemulsions, vesicles such as liposomes, tubes, rods, sheets, bilayers, nano/micro capsules, liquid crystals, and other self-assembled structures) are useful for creating microenvironment around the active agent that is preserved upon entering into different environment (e.g. when the active agent is released from the formulation into a high pH region in the GI tract). These self-assembling agents may be exemplified by lipids and derivatives, self-assembling amphiphilic molecules, self-assembling peptidic lipids, self-assembling block copolymers, self-assembling carbohydrate derivatives, dendrimers, polyions complexes, phospholipids and derivatives, glycolipids, polynucleotides, gemini surfactants, bile salts, etc.

In addition to adjusting the solubility of the active agent, it is also important to regulate the release profile of the drug from the dosage form, including the lag time prior to drug release. Compounds used to regulate the release in the practice of the current invention (“release regulating agents”) include, but are not limited to, slow dissolving materials, soluble polymers, gelling agents, hydrophobic materials, osmotic agents, especially lower solubility osmotic agents, and pH sensitive materials. To give some examples, the release regulating agents may be selected from hydroxypropyl cellulose, hydroxypropyl methylcellulose and hydroxyethyl cellulose, tocopheryl polyethylene glycol succinates, polymethacrylates such as Eudragit E100, gelatin, phospholipids (known for their slow hydration), polyethylene/maleic anhydride copolymers, non-swellable poly(ethylene oxide)s, non-swellable polyvinylpyrrolidones, polyvinyl alcohols, polyethylene glycols, polypropylene glycols, various carrageenans, alginic acid and salts, agar, fatty acids and salts and derivatives including esters, glycerol esters, glycerol behenate, magnesium stearate, calcium stearate, stearic acid, hydrogenated vegetable oils, fatty alcohols and derivatives, waxes, isomalt, osmotic polysaccharides such as dextrans and branched dextrans, chitosans, acrylic and methacrylic based polymers such as Eudragit L 100-55, Eudragit L 30-D55, Eudragit L 100, Eudragit S 100, Eudragit FS 30 D, cellulose-based pH sensitive materials such as cellulose acetate phthalate, carboxymethylethylcellulose, cellulose acetate trimellitate, hydroxypropylmethylcellulose phthalate, hydroxypropylmethylcellulose acetate succinate, Shellacs such as Emcoat 120N and Marcoat 125, and polyvinyl acetate phthalate. Some of these compounds may also used to control the solubility of the active ingredients; i.e. some of the above-mentioned compounds have a dual function of controlling the solubility and modifying the release of the active ingredient. In other cases, release regulating agents and solubility modifiers provide a synergistic influence on the release of the active agent, as, for example, in the case of such compounds as xylitol, mannitol, maltrin, and isomalt in combination with acrylic and methacrylic based polymers such as Eudragit L 100-55, Eudragit L 30-D55, Eudragit L 100, Eudragit S 100, Eudragit FS 30 D.

The solubility modulators, alone or in combination with the release regulating agents, can be utilized for the preparation of various dosage forms, such as osmotic tablets, matrix tablets, capsules, beads, granules, powders, caplets, troches, sachets, cachets, pouches, gums, sprinkles, solutions and suspensions of the active compounds.

Osmotic controlled release techniques utilize a semipermeable membrane to allow only water to penetrate through the membrane into the core. The pH inside the osmotic dosage form is independent of the outside pH, thus the solubility of the active compounds inside the osmotic dosage form is also independent of the outside pH. However, it is desirable to ensure that the solubility of the active compounds inside the osmotic dosage form remains at a certain operational level to obtain consistent dissolution, and is high enough to avoid precipitation of the active compounds at or near the orifice due to a sudden change in environmental pH.

The osmotic formulations comprise a core and a semipermeable membrane bearing at least one orifice of the size from about 70 microns to about 1000 microns. Preferably, the size of the orifice is from about 100 microns to about 800 microns. The active compounds are contained in the core of the osmotic formulation. The core may further comprise at least one osmotic agent and at least one solubility modulator. The core may be covered with a subcoat, and then with a semipermeable membrane. The semipermeable membrane is drilled with at least one orifice.

The semipermeable membrane, which surrounds the drug-containing core, comprises a water insoluble, pharmaceutically acceptable polymer. Suitable water insoluble polymers include, for example, cellulose esters, cellulose ethers and cellulose ester ethers. Examples of such polymers include cellulose acylate, cellulose ethyl ether, cellulose diacylate, cellulose triacylate, cellulose acetate, cellulose diacetate, cellulose triacetate, mono-, di- and tricellulose alkyls, mono-, di- and tricellulose aroyls and the like. Cellulose acetate is the preferred polymer. Other suitable water insoluble polymers are disclosed in U.S. Pat. Nos. 4,765,989 and 4,077,407, which are hereby incorporated in their entirety by reference, and can be synthesized by procedures described, for instance, in the Encyclopedia of Polymer Science and Technology, Vol. 3, pp. 325-354 (1964), Interscience Publishers Inc., New York, N.Y.

The water insoluble polymeric materials used for the semipermeable membrane are preferably combined with plasticizers to impart increased flexibility, durability, stability and water permeability to the semipermeable membrane. Plasticizers that can be used to impart flexibility and elongation properties to the semipermeable membranes include phthalate plasticizers, such as dibenzyl phthalate, dihexyl phthalate, butyl octyl phthalate, straight chain phthalates of six to eleven carbons, di-isononyl phthalate, di-isodecyl phthalate, and the like. The plasticizers also include non-phthalates such as triacetin, dioctyl azelate, epoxidized tallate, tri-isooctyl trimellitate, tri-isononyl trimellitate, sucrose acetate isobutyrate, epoxidized soybean oil, and the like.

In the case of cellulose acetate, examples of suitable plasticizers are triethyl citrate (TEC), propylene glycol (PG), a mixture of TEC and PG with the amounts of TEC ranging from 15% to 85%, Tween 80 or other polyoxyethylene sorbitan esters, triacetin, diethyl phthalate, mineral oil, tributyl sebacate, glycerol, polyethylene glycol (PEG) of various molecular weights (e.g., from 400 to 6000 g/mol), and mixtures of TEC and PEG with the amounts of TEC ranging from 15% to 85%.

The amount of plasticizer in the semipermeable wall, when incorporated therein, is typically from about 0.01% to 30% by weight, or higher. The subcoat located between the membrane and the core may be of a polymer that hydrates or dissolves over a finite time to produce a delay in the release of the drug. This polymer may be a soluble polymer selected from a group consisting of cellulosic polymers (for example, hydroxypropyl cellulose, hydroxypropyl methylcellulose), polyvinylpyrrolidones, polyethylene glycols, polyvinyl alcohols, and so on. Additionally, the semipermeable membrane may be covered by one or more additional coats. The additional coat can be an over-coat, a delayed-release coat, or a coat containing pharmaceutically active compounds or other suitable pharmaceutical ingredients, or combinations thereof.

The dosage forms of the present invention may comprise release regulating agents, which can be in the core, in the semipermeable membrane, or in the additional coats. In one embodiment, the release regulating agent provides a means of delaying the release of the active compounds in the gastric environment, thus potentially reducing certain related side effects and improving patient compliance.

Osmotic agents useful for the osmotic formulations of the current invention are well known in the art. Osmotic agents useful for the current invention include non-swellable compounds represented by, but not limited to, polyols; carbohydrates including monosaccharides, oligosaccharides, polysaccharides and sugar alcohols; salts; acids and hydrophilic polymers. For example, osmotic agents may be selected from mannitol, maltrin, xylitol, maltitol, lactitol, isomalt, sorbitol, arabitol, erythritol, ribitol, insositol, lactose, glucose, sucrose, raffinose, fructose, dextran, glycine, urea, citric acid, tartaric acid, sodium chloride, potassium chloride, magnesium chloride, disodium hydrogen phosphate, sodium phosphate, potassium phosphate, sodium sulfate, lithium sulfate, magnesium sulfate, magnesium succinate, polyethylene glycol, maltodextrin, cyclodextrins and derivatives, non-swelling block polymers of PEO and PPO, polyols, polyethylene glycols and cellulose ethers.

While some osmotic agents have a significant negative impact on the solubility of the active compound, the agents that are preferred for the practice of the current invention maintain or even enhance the solubility of the active compound. Such osmotic agents have multiple functions in the current invention acting as solubility modulators and/or as release regulating agents.

In one embodiment of the invention, the osmotic agents are such that they allow for a delayed release (lag time) of the active compounds by providing an appropriate osmotic pressure gradient. Examples of such osmotic agents include, but are not limited to, polyols or sugar alcohols such as isomalt (various grades of GalenIQ, e.g. GalenIQ 810, 800, 801, 720, 721 etc.).

The lag time before the release of the active compound can also be altered by adjusting the coating level and composition of the semipermeable membrane and the size of the orifice.

Other suitable excipients well known in the art, such as binders, lubricants, glidants, bulking agents, absorption enhancers, colorants, flavorants, stabilizers and taste-masking agents, can be also incorporated into the formulation to further improve the product attributes and processability. For example, bulk agents, such as microcrystalline cellulose, calcium phosphate, calcium carbonate, starch, etc., can be added to impart the bulk of the dosage form. Lubricants, such as magnesium stearate, stearic acid, etc., can be added to reduce the friction between the material and the equipment. Glidants and anti-adherents, such as silicon dioxide, corn starch, compritol 888 ATO, talc, etc., can be used to improve the flowability and reduce the sticking tendency of the formulation.

In one embodiment, the invention is a controlled release formulation of desvenlafaxine that provides consistent dissolution of the active compound. In particular, the current invention discloses controlled release formulations of (−)-O-desmethyl venlafaxine HCl monohydrate, where the solubility of the drug is stabilized in the range of from about 150 mg/mL to about 900 mg/mL for the duration of the release (i.e. throughout the complete range of GI pH values).

In a particular embodiment, the controlled release of desvenlafaxines and related active compounds is achieved through an osmotic dosage form. In this case, the osmotic dosage form comprises a desvenlafaxine, at least one solubility modulator, and optionally, an osmotic agent, as described above. Alternatively, in a specific embodiment when the osmotic pressure of the drug is sufficiently high, the osmotic dosage form may comprise a semipermeable membrane and a core that comprises the active agent and a processing aid but does not require the presence of the osmotic agent.

Specific examples of solubility modulators and release regulators useful for the osmotic dosage forms of the current invention include, but are not limited to, mannitol, sorbitol, maltodextrins (e.g. Maltrin M150), lactose, xylitol, isomalt (e.g. various grades of GalenIQ), sodium chloride, polyamines, polyvinyl amines, polyimines, polyamides, polyaminoacids/peptides, aminopolysaccharides such as chitosan, polyalginates, polyvinyl pyridines, polyvinylpyrrolidones (e.g. Kollidone), hydroxypropylcellulose (e.g. Klucel LF), hydroxypropyl methylcellulose (e.g. Methocel E5), cellulosic phthalates (e.g. hydroxypropylmethylcellulose phthalate), polyethylene glycols (various Mw., e.g. 3350 and 8000), citric acid, tartaric acid, malic acid, acrylic polymers (e.g. Eudragit L100, Carbopol 941), alginic acid, sodium lauryl sulfate, cyclodextrins (e.g. alpha-, beta-, gamma-, alkylated-, derivatized-cyclodextrins, for example, hydroxypropyl beta cyclodextrin, methyl cyclodextrin, sulfo-cyclodextrins, and so on), polyethylene glycol esters (e.g. Myrj 52S), sodium docusate, polysorbates (e.g. Tween 80), polyglycolized glycerides (e.g. Gelucire 44/14), block copolymers of ethylene oxide and propylene oxide (e.g. Poloxamer 188), vitamin E TPGS, fumaric acid, ascorbic acid, triethyl citrate, cholestyramine resin, polysaccharide-based anionic exchange resins, carboxy- or sulfonated polystyrene resins, oxa- and thia-crown ethers, polyoxoalkylenes, or polysiloxanes.

As stated above, the solubility of (−)-O-desmethyl venlafaxine HCl monohydrate is highly dependent on pH. At low pH, for example pH 1.0, the solubility is in the range of a few hundred mg/mL; at about pH 6.0, the solubility is less than 100 mg/mL; at higher pH, e.g. close to the physiological pH, the solubility dramatically decreases to less than 1 mg/mL. The stabilizing influence of the various modulators on the solubility of (−)-O-desmethyl venlafaxine HCl monohydrate is demonstrated in Table 1.

By way of an example, a saturated solution of (−) O-desmethyl venlafaxine HCl monohydrate was prepared by dissolving an excess amount of the active compound in the presence of solubility modulators. All samples were prepared with DI water, pH 5.5. Controls were prepared similarly without the use of modulators. All test samples were mixed overnight at room temperature. The samples were then filtered and analyzed using HPLC-UV method.

Table 1 shows the solubility of (−)-O-desmethyl venlafaxine HCl monohydrate in the presence of various solubility regulating agents.

TABLE 1

| | API | |
|---|---|---|
| | Solubility | |
| Name | mg/mL | Sample pH |
| Tartaric acid | 719 | 1.5-2.0 |
| Malic acid | 677 | 1.5-2.0 |
| Lactose | 665 | 5.2-5.5 |
| Kollidone/Tartaric acid | 661 | 2.1-2.4 |
| Triethyl citrate | 643 | 5.2-5.5 |
| Fumaric acid | 639 | <1.8 |
| Mannitol | 613 | 5.2-5.5 |
| Citric acid | 612 | 2.1-2.4 |
| Kollidone 25 | 610 | 5.2-5.5 |
| PEG 3350 | 602 | 5.2-5.5 |
| Ascorbic acid | 597 | 2.7-3.0 |
| Hydroxypropyl beta | 589 | 5.5-5.7 |
| cyclodextrin | | |
| Carbopol 941 | 587 | 3.2-3.5 |
| HPMCP | 572 | 2.7-3.0 |
| PEG 8000 | 569 | 5.2-5.5 |
| Alginic acid | 568 | 2.4-2.7 |
| Maltrin M150 | 565 | 5.2-5.5 |
| Methylcyclodextrin | 561 | 5.5 |
| Poloxamer 188 | 552 | 5.2-5.5 |
| Sorbitol | 549 | 5.2-5.5 |
| Xylitol | 547 | 5.2-5.5 |
| Eudragit L100 | 547 | 3.2-3.5 |
| Tween 80 | 544 | 5.2-5.5 |
| Sodium Docusate | 539 | 6.3-6.5 |
| Klucel LF | 536 | 5.5 |
| Vitamin E TPGS | 535 | 5.2-5.5 |
| Sodium lauryl sulfate | 524 | 6.3-6.6 |
| Gelucire 44/14 | 521 | 5.2-5.5 |
| Mannitol/Sorbitol/Tartaric | 521 | <1.8 |
| acid/Eudragit L100 | | |
| Methocel E5 | 504 | 5.7 |
| Beta cyclodextrin | 502 | 5.2-5.5 |
| Maltrin | 432 | 5.2-5.5 |
| M150/Xylitol/Sodium lauryl | | |
| sulfate/Gelucire 44/14 | | |
| API Control | 611 | 5.2-5.5 |

The following examples are presented to illustrate and do not limit the invention.

### EXAMPLES

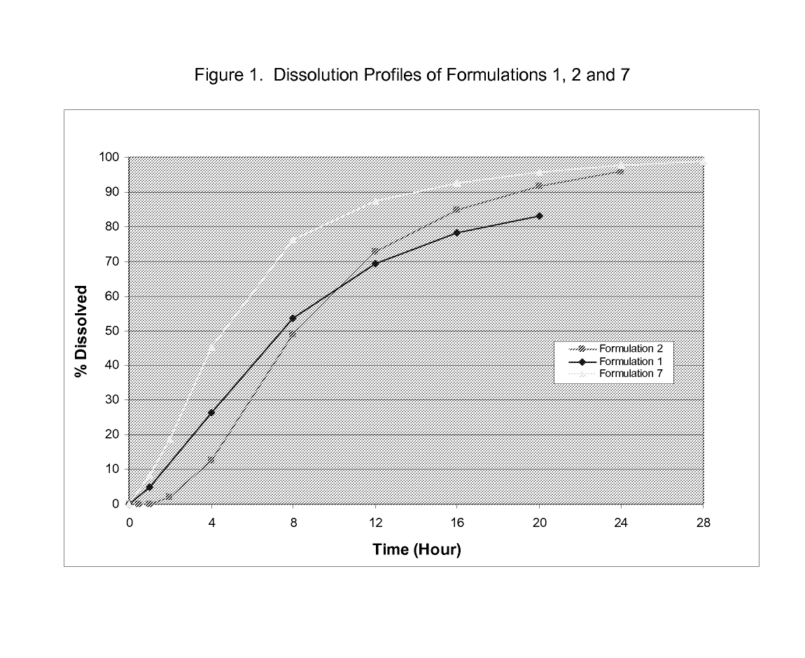

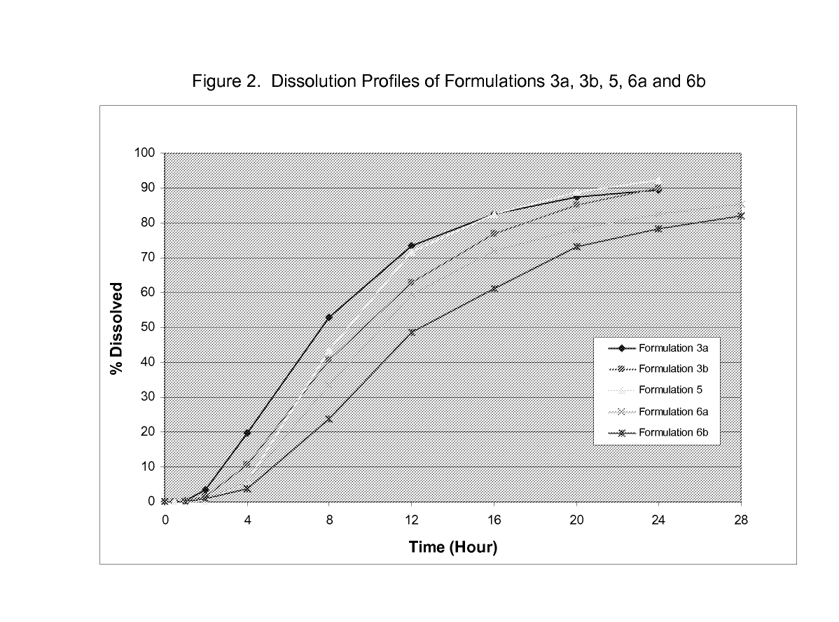

Non-exhaustive formulation examples are listed in Table 2 and assayed in FIGS. 1 and 2.

Table 2 provides weight percent compositions of (−)-O-Desmethyl Venlafaxine HCl Monohydrate core tablets.*

TABLE 2

| | Formulation | | | | | | |
|---|---|---|---|---|---|---|---|
| | | | 3a** | | | 6a** | |
| | | | and | | | and | |
| Ingredients | 1 | 2 | 3b** | 4 | 5 | 6b** | 7 |
| (−)-O-Desmethyl | 24.87% | 25.00% | 28.69% | 28.69% | 28.69% | 20.12% | 98.00% |
| Venlafaxine HCl | | | | | | | |
| Monohydrate | | | | | | | |
| Mannitol | — | 39.00% | 33.28% | — | 34.23% | 38.38% | — |
| Maltrin M150 | 37.80% | — | — | 31.57% | — | — | — |
| GalenIQ 810 | — | 30.00% | 27.10% | — | 28.05% | 32.00% | — |
| PVP K30 | — | 5.00% | 4.75% | — | — | — | — |
| PVP K25 | — | — | — | 4.75% | — | — | — |
| Klucel | — | — | — | — | 2.85% | 3.00% | — |
| SLS | 4.97% | — | — | — | — | — | — |
| Eudragit L100 | — | — | 2.85% | — | 2.85% | 3.00% | — |
| Avicel PH102 | — | — | — | — | — | — | 1.00% |
| Compritol 888 | — | — | 1.90% | 1.90% | 1.90% | 2.00% | — |
| ATO | | | | | | | |
| Magnesium | 0.53% | 1.00% | 1.43% | 1.43% | 1.43% | 1.50% | 1.00% |
| stearate | | | | | | | |

*The tablets at various target weights were coated with cellulose acetate in acetone containing triethyl citrate; then drilled by a laser to yield the final osmotic tablets.
**a and b are at different cellulose acetate coating levels.

### Example 1

(−)-O-desmethyl venlafaxine HCl monohydrate was formulated with solubility modulators and release regulating agents. A dry blend of sodium lauryl sulfate, xylitol, Maltrin M150 was fluidized in a fluid bed processor. A solution of the active compound and a binder (Maltrin M150) was sprayed onto the blend to form granules. The dried granules were screened through an 18-mesh screen and blended with a lubricant in a V-blender for a specific period of time. The final blend was then compressed into tablets with different tablet weights based on the formulation strength. These tablets were then coated with cellulose acetate in acetone containing a plasticizer (e.g. triethyl citrate) at 5-10% solids content. The targeted weight gain of the tablets after coating was typically 2% to 5% (Formulation 1, Table 2). The coated tablets were drilled by a laser with an appropriate mask to create an orifice of appropriate size (about 125 micron) for osmotic applications.

### Example 2

(−)-O-desmethyl venlafaxine HCl monohydrate was formulated with solubility modulators and release regulating agents. The active compound, mannitol and GalenIQ 810 were premixed in a bag for a specific time. The powder was then fluidized in the fluid bed processor. An aqueous solution of PVP K30 was then sprayed onto the powder to form granules. The dried granules were screened through an 18-mesh screen and blended with a lubricant (magnesium stearate) in a V-blender for a specific period time. The final blend was then compressed into tablets of varying dose strengths. These tablets were then coated with cellulose acetate in acetone containing a plasticizer (e.g. triethyl citrate) at 5-10% solids content. The targeted weight gain of the tablets after coating was typically 2% to 5% (Formulation 2, Table 2). The coated tablets were drilled by a laser with an appropriate mask for osmotic applications.

### Example 3

(−)-O-desmethyl venlafaxine HCl monohydrate was formulated with solubility modulators and release regulating agents. The active compound and Eudragit L100 were mixed in a bag for a specific period of time. A portion of mannitol was added to the bag and the powder was mixed again. The mixed powder, GalenIQ 810 and the remaining mannitol were charged and fluidized in a fluid bed processor. An aqueous solution of PVP K30 was then sprayed onto the powder to form granules. The dried granules were screened through an 18-mesh screen and blended with a glidant (Compritol 888 ATO), then with a lubricant (magnesium stearate) in a V-blender for specific periods of time. The final blend was compressed into tablets with different tablet weights based on the formulation strength. These tablets were then coated with cellulose acetate in acetone containing a plasticizer (e.g. triethyl citrate) at 5-10% solids content. The targeted weight gain of the tablets after coating was typically 2% to 5% (Formulation 3a and 3b, coated at different levels, Table 2). The coated tablets were drilled by a laser with an appropriate mask for osmotic applications.

### Example 4

(−)-O-desmethyl venlafaxine HCl monohydrate was formulated with solubility modulators and release regulating agents. The active compound, xylitol and Maltrin M150 were charged and fluidized in a fluid bed processor. An aqueous solution of PVP K25 was then sprayed onto the powder to form granules. The dried granules were screened through an 18-mesh screen and blended with a glidant (Compritol 888 ATO), then with a lubricant (magnesium stearate) in a V-blender for specific periods of time. The final blend was compressed into tablets with different tablet weights based on the formulation strength. These tablets were then coated with cellulose acetate in acetone containing a plasticizer (e.g. triethyl citrate) at 5-10% solids content. The targeted weight gain of the tablets after coating was typically 2% to 5% (Formulation 4, Table 2). The coated tablets were drilled by a laser with an appropriate mask for osmotic applications. Two different lots of tablets were produced: one with an orifice size of 230 micron (lot 4a), and the other with an orifice size of 430 micron (lot 4b).

### Example 5

(−)-O-desmethyl venlafaxine HCl monohydrate was formulated with solubility modulators and release regulating agents. The active compound and Eudragit L100 were mixed in a bag for a specific period of time. A portion of mannitol was added to the bag and the powder was mixed again. The mixed powder, GalenIQ 810 and the remaining mannitol were charged and fluidized in a fluid bed processor. An aqueous solution of Klucel (for example, Klucel EXAF) was then sprayed onto the powder to form granules. The dried granules were screened through an 18-mesh screen and blended with a glidant (Compritol 888 ATO), then with a lubricant (magnesium stearate) in a V-blender for specific periods of time. The final blend was compressed into tablets with different tablet weights based on the formulation strength. These tablets were then coated with cellulose acetate in acetone containing a plasticizer (e.g. triethyl citrate) at 5-10% solids content. The targeted weight gain of the tablets after coating was typically 2% to 5% (Formulation 5, Table 2). The coated tablets were drilled by a laser with an appropriate mask for osmotic applications.

### Example 6

(−)-O-desmethyl venlafaxine HCl monohydrate was formulated with solubility modulators and release regulating agents. The active compound and Eudragit L100 were mixed in a bag for a specific period of time. A portion of mannitol was added to the bag and the powder was mixed again. The mixed powder, GalenIQ 810 and the remaining mannitol were charged and fluidized in a fluid bed processor. An aqueous solution of Klucel (for example, Klucel EXF) was then sprayed onto the powder to form granules. The dried granules were screened through an 18-mesh screen and blended with a glidant (Compritol 888 ATO), then with a lubricant (magnesium stearate) in a V-blender for specific periods of time. The final blend was compressed into tablets with different tablet weights based on the formulation strength. These tablets were then coated with cellulose acetate in acetone containing a plasticizer (e.g. triethyl citrate) at 5-10% solids content. The targeted weight gain of the tablets after coating was typically 2% to 8% (Formulations 6a and 6b, Table 2). The coated tablets were drilled by a laser with an appropriate mask for osmotic applications.

### Example 7

(−)-O-desmethyl venlafaxine HCl monohydrate was formulated without solubility modulators. The active compound and a small amount of bulking agent (Avicel PH102) were mixed in a bag for a specific period of time. The powder was then mixed with a lubricant (magnesium stearate) for specific periods of time. The final blend was compressed into tablets with different tablet weights based on the formulation strength. These tablets were then coated with cellulose acetate in acetone containing a plasticizer (e.g. triethyl citrate) at 5-10% solids content. The targeted weight gain of the tablets after coating was typically 1% to 8% (Formulation 7, Table 2). The coated tablets were drilled by a laser with an appropriate mask for osmotic applications.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

What is claimed is:

1. A controlled release pharmaceutical formulation with a consistent release profile comprising a mixture of: (1) at least one pharmaceutical agent with a pH-dependent solubility; and (2) at least one solubility modulator.

2. The formulation of claim 1, wherein the solubility modulator is selected from a group consisting of complexing agents, buffering agents, acids and/or salts thereof; acid-base complexes, acidic polymers, acrylic acid polymers and copolymers, cationic polymers, ion exchange resins, lipids including phospholipids and their analogues and derivatives, ionic surfactants, peptides selected from oligo- and polypeptides, carbohydrates selected from cationic or non-ionic carbohydrates, polycarbohydrates and oligocarbohydrates; non-ionic surfactants, non-ionic polymers with donor/receptor functional groups, poly(γ-benzyl L-glutamate), lactide and glycolide polymers and copolymers and their derivatives, polyethyleneglycol-poly(ortho ester) block copolymers, poly(styrene)-poly(methylmethacrylate) block copolymers, PEO-PPO-PEO block copolymers, poly(2-phenoxyethyl vinyl ether)-poly(2-methoxyethyl vinyl ether)) block copolymers, carbohydrate-substituted porphyrins, block polymers comprising polyacetylene, block polymers comprising poly(p-phenylene vinylene), block polymers comprising poly(p-phenylene), block polymer comprising polypyrrole, block polymers comprising polythiophene, and alginic acid complexes.

3. The formulation of claim 2, wherein the solubility modulator creates a microenvironment around the active agent by forming micro- or nano-structures selected from micelles, reverse micelles, microemulsions, vesicles such as liposomes, tubes, rods, sheets, bilayers, nano/micro capsules, and liquid crystals.

4. The formulation of claim 3, wherein said solubility modulator is selected from a group consisting of lipids and derivatives, self-assembling amphiphilic molecules, self-assembling peptidic lipids, self-assembling block copolymers, self-assembling carbohydrate derivatives, dendrimers, polyions complexes, phospholipids and derivatives, glycolipids, polynucleotides, gemini surfactants, and bile salts.

5. The formulation of claim 1, wherein the solubility regulating agent is selected from a group consisting of mannitol, sorbitol, isomalt, maltodextrins, lactose, xylitol, polyethylene glycol, polyvinyl pyrrolidones, polyvinyl alcohols, polyamines, polyvinyl amines, polyimines, polyamides, polyaminoacids/peptides, aminopolysaccharides, chitosan, polyalginates, polyvinyl pyridines, hydroxypropyl cellulose, hydroxypropyl methyl cellulose, hydroxypropylmethyl cellulose phthalate, citric acid, tartaric acid, malic acid, fumaric acid, ascorbic acid, methacrylic acid copolymers, Eudragit L100, alginic acid, sodium lauryl sulfate, hydroxypropyl beta cyclodextrin, beta cyclodextrin, alpha cyclodextrin, gamma cyclodextrin, methylcyclodextrin, sulfated cyclodextrins, sulfated substituted cyclodextrins, sodium docusate, polysorbates, polyglycolized glycerides, Gelucire 44/14, ethylene oxide and propylene oxide block copolymers, poloxamer 188, cross-linked acylic acid polymers, Carbopol 941, triethyl citrate, D alpha tocopheryl polyethylene glycol 1000 succinate, polyethylene glycol esters, Myrj 52S, cholestyramine resin, carboxy- or sulfonated polystyrene resins, oxa- and thia-crown ethers, polyoxoalkylenes, and polysiloxanes.

6. The formulation of claim 1 further comprising a release-regulating agent selected from the group consisting of slow dissolving materials, soluble polymers, gelling agents, hydrophobic materials, osmotic agents especially lower solubility osmotic agents, and pH sensitive materials.

7. The formulation of claim 6 in which the pH sensitive material is an acrylic polymer.

8. The formulation of claim 1 which in the form of a dosage form selected from osmotic tablets, matrix tablets, capsules, beads, granules, powders, caplets, troches, sachets, cachets, pouches, gums, sprinkles, solutions and suspensions.

9. The formulation of claim 1 wherein the at least one excipient is selected from binders, lubricants, glidants, bulking agents, absorption enhancers, colorants, flavorants, stabilizers and taste-masking agents.

10. The formulation of claim 1, which is a matrix formulation.

11. The formulation of claim 1, which is an osmotic formulation.

12. The formulation of claim 11, comprising a pharmaceutical agent-containing core and a semipermeable membrane comprising at least one orifice.

13. The formulation of claim 12, further comprising a release delaying subcoat located between the semipermeable membrane and the core.

14. The formulation of claim 11, further comprising a coating on top of the semipermeable membrane.

15. The formulation of claim 14, wherein the coating is an over-coat, a delayed-release coat, or a coat containing one or more active compounds or other suitable active pharmaceutical ingredients.

16. The formulation of claim 12, wherein the core comprises the pharmaceutical agent, the solubility modulator and at least one osmotic agent.

17. The formulation of claim 16, wherein the osmotic agent is selected from polyols; carbohydrates, sugar alcohols; salts; acids and hydrophilic polymers.

18. The formulation of claim 16 wherein the carbohydrates are selected from monosaccharides, oligosaccharides, and polysaccharides.

19. The formulation of claim 16, wherein the osmotic agent is selected from polyethylene glycol, maltodextrins, cyclodextrins, polyglycerols, and polyelectrolytes.

20. The formulation of claim 16, wherein the osmotic agent is mannitol, xylitol, maltitol, lactitol, isomalt, sorbitol, arabitol, erythritol, ribitol, insositol, lactose, glucose, sucrose, raffinose, fructose, dextran, glycine, urea, citric acid, tartaric acid, sodium chloride, potassium chloride, magnesium chloride, disodium hydrogen phosphate, sodium phosphate, potassium phosphate, sodium sulfate, lithium sulfate, magnesium sulfate, magnesium succinate, polyethylene glycol, maltodextrin, cyclodextrins and derivatives, non-swelling block polymers of PEO and PPO.

21. The formulation of claim 16, wherein the osmotic agent simultaneously acts as the solubility modulator.

22. The formulation of claim 16, wherein the osmotic agent is a sugar alcohol.

23. The formulation of claim 22, wherein the osmotic agent is isomalt.

24. The formulation of claim 23, wherein the pharmaceutical agent is desvenlafaxine.

25. The formulation of claim 24, wherein the desvenlafaxine is (−)-O-desmethyl venlafaxine HCl monohydrate.

26. The formulation of claim 25, wherein the solubility of the (−)-O-desmethyl venlafaxine HCl monohydrate in the formulation is preserved in the range of from 150 mg/mL to 900 mg/mL throughout the release.

27. The formulation of claim 26 comprising polyvinyl pyrrolidone as the binder.

28. The formulation of claim 27, wherein the solubility regulating agent is Eudragit L100.

29. An osmotic formulation of desvenlafaxine comprising a semipermeable membrane and a core, wherein the core comprises from 1% wt to 70% wt of desvenlafaxine; from 1% wt to 50% wt of a solubility modulator selected from a group consisting of xylitol, mannitol, maltrin, isomalt, Eudragit L-100, and a binder selected from polyvinyl pyrrolidone and hydroxypropyl cellulose.

30. The osmotic formulation of claim 29, wherein the desvenlafaxine is a single enantiomer (−)-O-desmethyl venlafaxine HCl monohydrate.

31. An osmotic formulation of desvenlafaxine comprising a semipermeable membrane and a core, wherein the core comprises (−)-O-desmethyl venlafaxine HCl monohydrate and at least one pharmaceutically acceptable excipient.
